(19) United States
(12) Patent Application Publication
(10) Pub. No.: US 2023/0218920 A1
(43) Pub. Date: Jul. 13, 2023

(54) RADIATION TREATMENT DEVICE

(71) Applicant: I-SMART DEVELOPMENTS LIMITED, Wylde Green (GB)

(72) Inventor: SUSAN D'ARCY, Wylde Green (GB)

(21) Appl. No.: 17/928,143

(22) PCT Filed: May 28, 2021
(86) PCT No.: PCT/GB2021/051326

§ 371 Date: Nov. 28, 2022

(30) Foreign Application Priority Data
May 28, 2020 (GB) 2008034.7

(51) Int. Cl. A61N 5/06; A61N 5/067

(52) U.S. Cl. CPC A61N 5/0603; A61N 5/067; A61N 2005/0651; A61N 2005/0645; A61N 2005/0607; A61N 2005/0654; A61N 2005/0659

(57) ABSTRACT

A radiation treatment device comprises a radiation source layer comprising a radiation source and a controller. The radiation source is configured to emit radiation with a wavelength in the range 822 nm to 1322 nm. In a preferred embodiment, the device configured to emit radiation with a wavelength of 1067 to 1077 nm.

This invention relates to a light treatment device and an associated method of using a light treatment device.

### BACKGROUND

Photobiomodulation (PBM) therapy, formerly known as low-level light/laser therapy (LLLT), is the use of red and infrared (IR) light or radiation on the body to stimulate responses in cells and tissues. During PBM therapy, a treatment subject is exposed to light of a particular wavelength of radiation at an optimised dose. Light may then be absorbed by the chromophores of living tissue (for example, cytochrome C oxidase in the respiratory electron chain of mitochondria), which may result in the modulation of various cellular functions.

There are many known uses of PBM therapy, such as the treatment of musculoskeletal injuries and disorders, wound care and the alleviation of acute and chronic pain. Recent studies have suggested that IR radiation can be used to treat an array of diseases, including dermatologic conditions, Alzheimer's disease, Parkinson's disease, age-related macular degeneration, photo-ageing and alopecia areata, and that it can reduce the duration of herpes simplex labialis (HSL) lesions. It has been suggested that near infrared light can accelerate wound healing, possibly via upregulation of transforming growth factor beta 1 (TGF-β1) and matrix metalloproteinase-2 (MMP-2).

Since the onset of the COVID-19 pandemic, evidence has emerged that PBM therapy, particularly using red and near infrared radiation, may be a suitable intervention for subjects with COVID-19 infection. For example, it has been shown by Lim et al. that irradiation of the nasal cavity with a red light emitting diode (LED) emitting radiation with a 633 nm wavelength at a power density of 6.5 mW/cm² can inhibit coronavirus infection (see “Can the Vielight X-Plus be a Therapeutic Intervention for COVID-19 Infection”, Lew Lim, Michael R Hambin, 7 Apr. 2020).

One limitation of using radiation with a wavelength of 633 nm is that its penetration through the tissue being irradiated can be lower than, for example, radiation with a wavelength of 810 nm. This means that the intensity of the red light must be increased in order to increase penetration, leading to possible unwanted side effects such as skin warming. More powerful and more expensive apparatus may also be required.

Lim et al. also suggest providing PBM therapy using infrared light with a wavelength of 810 nm by positioning an LED module emitting light with a wavelength of 810 nm on the sternum of the subject, with the selected wavelength being recognised for its improved penetration into tissue compared to a wavelength of 633 nm.

In viral infections, the host response and clearance of the virus relies heavily on type I interferons (I IFNs). Expression of I IFNs initiates cell-mediated immune responses, promoting infection control and pathogen clearance.

It has been shown that irradiation at a wavelength of 1072 nm may have significant PBM effects. S Y Celine Lee et al. inoculated mice with MRSA and them exposed to radiation at a wavelength of 1072 nm (S. Y. Celine Lee et al., Enhancement of cutaneous immune response to bacterial infection after low-level light therapy with 1072 nm infrared light: A preliminary study, J. Photochem. Photobiol. B: Biol. (2011), doi:10.1016/j.jphotobio1.2011.08.009). FIG. 1 illustrates the relative proportions of the mRNA levels of measured cytokines and growth factors, and hence their level of transcription, for mice treated with radiation having a wavelength of 1072 nm compared to mice in a control group treated with sham light.

In the study, interleukin 1β (IL-1β) showed more sustained increasing tendency than toll like reception 2 (TLR2), its mRNA level being twice as high as that of the control group 12 h from inoculation. It further increased as high as 23 times, 27 times and 22 times compared to the control group after 24 h, 3 days, and 5 days respectively after inoculation.

Interleukin 6 (IL-6), which is known to increase in the acute phase of inflammation, also showed an enhanced response compared to the control. It was three times as high as the control at 1 h post inoculation and increased up to 17 times the level of the control 12 h post-inoculation. The IL-6 mRNA level was normalised to the control level at 24 h after the inoculation (1.9 times of the level of the control).

Nuclear translocation of nuclear factor kappa-light-chain-enhancer of activated B cells (NF-κB) represents the activation and initiation of transcription genes that are involved in the rapid response to bacterial infection. Nuclear translocation of NF-κB was markedly observed as early as 12 h after the inoculation in the 1072 nm group, whereas this feature was not found at this timepoint in the control group. In the control group, the nuclear transcription was observed at day 3.

Tumour necrosis factor alpha (TNF-α), another primary cytokine, along with IL-1β also showed remarkable increases starting from as early as 4 h post-inoculation compared to the control. The mRNA levels of TNF-α continued to increase, peaking at 24 h after the inoculation (26 times higher compared to the control) and normalised again to the control level at 3 days after the inoculation.

Of great importance to the present invention is that iNOS, the inducible form of nitric oxide synthase, started increasing 24 h from inoculation. It was 1.7 times as high as the control group at 24 h and 3 days after the inoculation and further increased to 2.7 times after 5 days.

Nitric oxide (NO) is an important signalling molecule between cells which has been shown to have an inhibitory effect on some virus infections, and iNOS may have an inhibitory effect on the replication cycle of the SARS CoV virus.

Near IR radiation may increase NO production by the mitochondria of the cells at the site being irradiated. NO is known to regulate apoptosis in varying types of cells and may promote or antagonise apoptosis depending on the type of cell and the concentration of NO present. NO also has antimicrobial activity against bacteria and fungi and is known to have antiviral activity against some viruses.

Another important benefit of the 1072 nm wavelength is its increased penetration depth; a wavelength of 1072 nm has been clinically proven to penetrate soft tissue, bon and brain parenchyma.

The present invention seeks to alleviate the disadvantages of the prior art by providing an improved light treatment device. The device may be used for the treatment of COVID-19 and for the maintenance or improvement of organic or body tissues, including muscles. The invention may be used in connection with the cure or alleviation of a variety of diseases including infectious diseases and pathological processes including those caused by viruses and bacteria.

### SUMMARY OF THE INVENTION

According to the invention there is provided a radiation treatment device comprising:
a radiation source layer comprising a radiation source; and
a controller;
   wherein the radiation source is configured to emit radiation with a wavelength in the range 822 nm to 1322 nm.

The radiation source may be configured to emit radiation with a wavelength in the range 922 to 1222 nm.

The radiation source may be configured to emit radiation with a wavelength in the range 1022 to 1122 nm.

The radiation source may be configured to emit radiation with a wavelength in the range 1047 to 1097 nm.

The radiation source may be configured to emit radiation with a wavelength in the range 1062 to 1082 nm.

The radiation source may be configured to emit radiation with a wavelength in the range 1067 to 1077 nm.

The irradiance of the radiation source may be in the range 10 mW/cm² to 10 W/cm² and may preferably be in the range 50 mW/cm² to 2 W/cm².

The radiation source may comprise a light emitting diode, a xenon lamp or a laser diode.

The radiation treatment device may comprise a microprocessor for controlling the radiation source, and may comprise a user interface which is connected to the microprocessor.

The radiation treatment device may comprise a backing layer, which may comprise a flexible material.

The backing layer may comprise or may further comprise an inflexible material.

The radiation treatment device may comprise attachment means for attaching the device to a subject's skin.

The radiation treatment device may comprise a wearable device.

The radiation treatment device may comprise air channels for allowing air flow through the device.

The radiation treatment device may comprise an intranasal device.

The radiation treatment device may comprise at least one sensor to detect at least one of the subject's respiratory rate, respiratory effort, blood pressure, heart rate, body temperature or blood oxygen saturation.

According to a second aspect of the invention there is provided a method of using the radiation treatment device, comprising the steps of applying the radiation treatment device to a subject and activating the radiation source to emit radiation.

The method may comprise the steps of selecting parameters for operation of the radiation source, inputting the parameters for operation of a radiation source into the control unit of the radiation treatment device and activating the radiation source to emit radiation in accordance with the input parameters.

According to a third aspect of the invention there is provided a therapeutic method comprising the use of the device. The method may be used for the treatment of viral infections such as SARS-CoV-2 infection.

According to a fourth aspect of the invention there is provided a therapeutic method comprising the method of using the radiation treatment device, comprising the steps of applying the radiation treatment device to a subject and activating the radiation source to emit radiation.

The therapeutic method may be used for the treatment of viral infections such as SARS-CoV-2 infection.

### BRIEF DESCIPTION OF DRAWINGS

FIG. 1 is a graph showing levels of measured cytokines and growth factors for mice inoculated with MRSA and treatment with radiation at a wavelength of 1072 nm (S. Y. Celine Lee et al., Enhancement of cutaneous immune response to bacterial infection after low-level light therapy with 1072 nm infrared light: A preliminary study, J. Photochem. Photobiol. B: Biol. (2011), doi:10.1016/j.jphotobio1.2011.08.009).

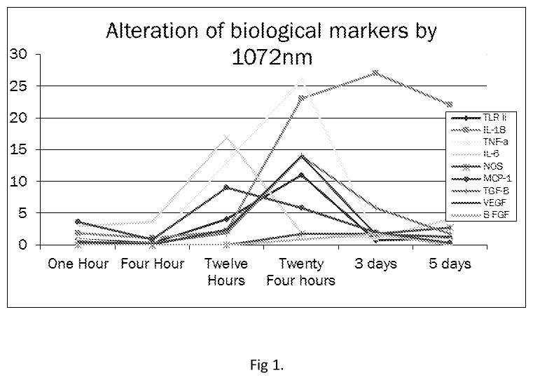

FIG. 2 is a schematic diagram of a light treatment device according to an embodiment the invention.

FIG. 3 is a schematic diagram of a method of using the light treatment device according to an embodiment of the invention.

FIG. 4 is a schematic diagram of a method of using the light treatment device according to a further embodiment of the invention.

### DETAILED DESCRIPTION

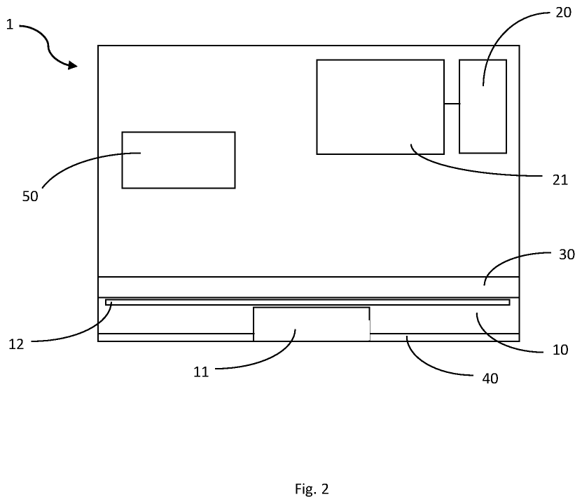

Referring to FIG. 2, there is shown a schematic diagram of a light treatment device 1 for treatment of a subject. The term “light” is not intended to confer any limitations of the wavelength of radiation emitted by the light treatment device. For the purposes of the specification, references to light are also intended to include references to radiation. The light treatment device 1 comprises a light source layer 10, a controller 20, a backing layer 30 and an adhesive layer 40.

The light source layer 10 comprises a light source 11. The light source 11 may be any suitable light source, for example an LED, xenon lamp or laser diode. In the illustrated embodiment, the light source layer 10 comprises a single light source 11, but it is envisaged that a plurality of light sources 11 may be provided. Where there is a plurality of light sources 11, these may be configured so that the light emitted by a light source 11 overlaps with an adjacent light source 11.

The light source 11 may be configured to emit light with a wavelength between 400 nm and 1600 nm with an irradiance of between 10 mW/cm² and 10 W/cm². In a preferred embodiment, the light source comprises multiple LEDs, each configured to emit a wavelength of between 1050 nm and 1300 nm with an irradiance of between 50 mW/cm² and 2 W/cm². In some embodiments, the light source 11 may be configured to provide an output period of irradiation of between 1 ms and 100 ms, and a repetition rate of between 50 Hz and 2500 Hz for a duration of between 1 second and 100 seconds.

In a more preferred embodiment, the light treatment device 1 emits a wavelength of about 1072 nm or 1268 nm at an intensity of 50 mW/cm² to 2 W/cm² using an LED, and the period of irradiation is a minimum of 10-15 ms with a repetition rate of 450 Hz and 800 Hz for a duration of at least 30 seconds.

The light source layer 10 of the illustrated embodiment also comprises a circuit board 12 which may be a flexible printed circuit board. The circuit board is adapted to connect the light source layer 10 to a power source 50 to provide power to the light source layer 10. In alternative embodiments, the backing layer 30 may comprise a or the circuit board. The power source 50 may comprise a battery, which may be a rechargeable battery. In other embodiments, the power source may comprise an electrical connector adapted to connect the light source layer 10 directly or indirectly to an external power supply. The light source layer may comprise a flexible silicone material. The silicone or other suitable alternative material may have a light dispersive effect.

The backing layer 30 may host the electrical components of the light treatment device 1. In some embodiment, the backing layer may be the rear of a printed circuit board or may comprise a circuit board, which may be a printed circuit board. In other embodiments, the backing layer may be a generic substrate upon which quantum dots are printed. In addition or alternatively, the light source layer 10 and the light source or sources 11 may comprise the quantum dots.

The backing layer 30 may be made from any suitable material and is preferably flexible. In a preferred embodiment, the backing layer comprises at least one flexible silicone material, although any other flexible material could be used such as elastomeric materials, cellulosic or aramid filter paper (such as the fibrous materials manufactured by Tyvek® and 3M® or film or thin gauze sheets made from materials such as polypropylene, polyethylene or polycarbonate. The backing layer may comprise a flexible laminate comprising two or more materials as a composite and may comprise a hydrogel. In other embodiments, the backing 30 layer may comprise a combination of flexible material and no flexible material combined to allow flexibility of the backing layer 30 in at least one orientation.

When the backing layer 30 and/or the light source layer 10 comprises a silicone material, the light source 11 may be an LED embedded in the silicone material. The silicone may provide a light-dispersive effect.

The light treatment device 1 comprises a controller 20 for user control of the light treatment device 1. The controller 20 may comprise an electrical switch for connecting and disconnecting the power supply from the power source 50 to the light source layer 10. In the illustrated embodiment, the light treatment device 1 comprises a microprocessor 21 and the controller 20 comprises a user interface which allows a user to select the parameters of the light to be emitted. For example, a user may select the desired wavelength, irradiance, pulse characteristics, output period, repetition rate and/or the duration for which light is emitted. The user interface may comprise an LED or LCD screen which may display details of selected or pre-selected parameters. In other embodiment, the interface may also display parameters such as, for example, battery charge information when the light treatment device comprises a rechargeable battery.

In an embodiment, the microprocessor 21 may be pre-programmed with pre-selected parameters to provide an output period of irradiation of between 1 ms and 100 ms and a repetition rate of between 50 Hz.

The controller may be connected to the power supply and/or the light source layer 10 via a wired connection. Alternatively, the controller may be connected to the power supply and/or the light source later via a wireless connection, which may comprise a radio connection means.

In an alternative embodiment, a wireless connection may allow the light treatment device 1 to be controlled by a separate user device, such as a PC or smartphone.

The light treatment device may comprise at least one sensor for measuring respiratory rate, respiratory effort, blood pressure, heart rate, body temperature and/or blood oxygen saturation. In embodiments comprising such a sensor, the user interface and microprocessor may be adapted to communicate with the at least one sensor. The at least one sensor may be configured to communicate with a separate user device, such as a PC or smartphone.

The light treatment device 1 may be configured to be worn on or by a subject. The light treatment device 1 may be adhered to the skin of the treatment subject using the adhesive layer 40. The adhesive layer 40 may comprise a substantially flat patch and may comprise a hydrogel. Alternatively, the light treatment device 1 may be attached to a subject using straps which fasten around the subject's body or may comprise a garment such as a vest or neckband. In an alterative embodiment, the light treatment device 1 may comprise an intranasal probe for insertion into the subject's nasal passage.

In some embodiments, the light treatment device 1 may comprise means for allowing regulation or cooling of the subject's skin, such as airways or forced air conduits.

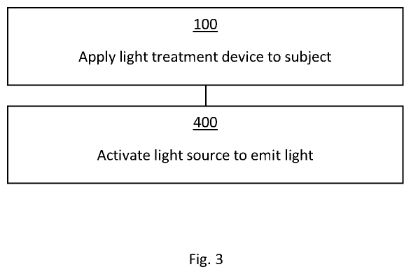

FIG. 3 shows a method of using an embodiment of the light treatment device 1. At step 100, the light treatment device is applied to the subject by the operator. (The operator may also be the treatment subject.) The device may be applied to any part of the body which is suitable for treatment with light, including the skin or the nasal passage. At step 400, the operator activates the light source to emit light directly to the treatment area. In this embodiment, the light treatment device 1 may be pre-programmed to emit light within predetermined parameters. While FIG. 3 shows the light treatment device 1 being applied to the subject before the light source 11 is activated, it is possible for these steps to be reversed.

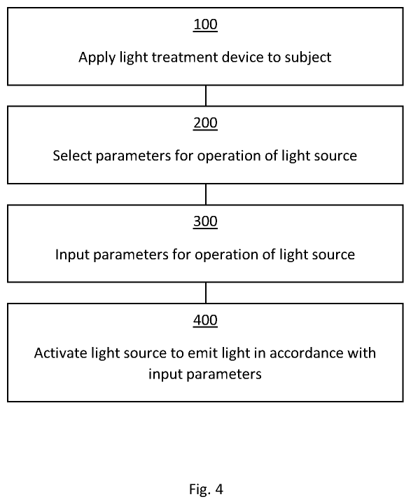

FIG. 4 shows an alternative method of using an embodiment of the light treatment device 1. In this embodiment, the light treatment device comprises a microprocessor and user interface. The operator applies the light treatment device to the subject at step 100. At step 200, the parameters for operation of the light treatment device, such as wavelength and intensity, may be selected by the operator and input into the device at step 300. The light source may then be activated to deliver the required light therapy to the subject. Again, the steps need not take place in this order and steps 200, 300 and/or 400 may be executed before step 100.

1. A radiation treatment device comprising:
a radiation source layer comprising a radiation source; and
a controller;
wherein the radiation source is configured to emit radiation with a wavelength in the range 822 nm to 1322 nm.

2. (canceled)

3. (canceled)

4. A radiation treatment device according to claim 1 wherein the radiation source is configured to emit radiation with a wavelength in the range 1047 to 1097 nm.

5. A radiation treatment device according to claim 4 wherein the radiation source is configured to emit radiation with a wavelength in the range 1062 to 1082 nm.

6. A radiation treatment device according to claim 5 wherein the radiation source is configured to emit radiation with a wavelength in the range 1067 to 1077 nm.

7. A radiation treatment device according to claim 1 wherein an irradiance of the radiation source is in the range 10 mW/cm² to 10 W/cm².

8. A radiation treatment device according to claim 1 wherein an irradiance of the radiation source is in the range 50 mW/cm² to 2 W/cm².

9. A radiation treatment device according to claim 1 wherein the radiation source comprises a light emitting diode.

10. A radiation treatment device according to claim 1 wherein the radiation source comprises a xenon lamp.

11. A radiation treatment device according to claim 1 wherein the radiation source comprises a laser diode.

12. A radiation treatment device according to 1 comprising a microprocessor for controlling the radiation source.

13. (canceled)

14. A radiation treatment device according to claim 1 further comprising a backing layer.

15. A radiation treatment device according to claim 14 wherein the backing layer comprises a flexible material.

16. A radiation treatment device according to claim 14 wherein the backing layer comprises or further comprises an inflexible material.

17. A radiation treatment device according to claim 1 wherein the device comprises attachment means for attaching the device to a subject's skin.

18. A radiation treatment device according to claim 1 wherein the device comprises a wearable device.

19. A radiation treatment device according to claim 1 wherein the device comprises air channels for allowing air flow through the device.

20. A radiation treatment device according to claim 1 wherein the device comprises an intranasal device.

21. A radiation treatment device according to claim 1 further comprising at least one sensor to detect at least one of the subject's respiratory rate, respiratory effort, blood pressure, heart rate, body temperature or blood oxygen saturation.

22. A method of using radiation treatment device, the radiation treatment device comprising a radiation source layer comprising a radiation source, and a controller, wherein the radiation source is configured to emit radiation with a wavelength in the range 822 nm to 1322 nm, the method comprising the steps of:
applying the radiation treatment device to a subject; and
activating the radiation source to emit radiation.

23. A method according to claim 22 further comprising the steps of:
selecting parameters for operation of the radiation source
inputting the parameters for operation of a radiation source into the control unit of the radiation treatment device; and
activating the radiation source to emit radiation in accordance with the input parameters.

24.-29 (canceled)
